(19) United States
(12) Patent Application Publication
(10) Pub. No.: US 2010/0228121 A1
(43) Pub. Date: Sep. 9, 2010

(54) Dual Chamber Syringe

(76) Inventors: Katada Kazuhiro, Aichi (JP); Munehito Kurimoto, Shizuoka (JP)

Correspondence Address: Mallinckrodt Inc., 675 McDonnell Boulevard, HAZELWOOD, MO, 63042, US

(21) Appl. No.: 11/993,030

(22) PCT Filed: Jun. 30, 2006
(86) PCT No.: PCT/US06/26452

§ 371 Date: Dec. 19, 2007

Related U.S. Application Data
(60) Provisional application No. 60/695,467, filed on Jun. 30, 2005.

(51) Int. Cl. A61B 6/00; A61M 5/20

(52) U.S. Cl. 600/432

(57) ABSTRACT

A dual chamber syringe (10) comprises an inner cylindrical body (30) having a open end, a closed end with an inner discharge outlet (35) formed therein, and an inner plunger (91) sealingly slideable within the inner body, and an outer cylindrical body (11) enclosing the inner cylindrical body and having an open end, a closed end with an outer discharge outlet (20) formed therein, and an outer plunger (75) sealingly slidable between the outer body and the inner body, the inner discharge outlet extending into the outer discharge outlet such that the inner body is in fluid communication through the outer discharge outlet with the exterior of the inner and outer body.

### FIELD OF THE INVENTION

The present invention relates to a dual chamber syringe for the injection of contrast media by an injector.

### BACKGROUND OF THE INVENTION

Syringes are typically used in medical applications to hold fluids for injection into patients. In some applications such as medical imaging using contrast media, the syringe is engaged to a power injector for driving the syringe plunger to inject fluid at a desired flow rate and/or pressure as needed for the medical procedure.

Frequently multiple liquids must be injected in a procedure, such as in a CT, Angiography, Ultrasound or other medical imaging procedure where injection of contrast media for medical imaging is followed and/or preceded by injection of saline solution. Similar multiple liquid injections may also appear in nuclear medicine or other applications.

For such applications, frequently the art has utilized a dual-head injector, having two syringes and two injector drives for driving the respective plungers in each of the dual syringes. This approach, however, raises various difficulties, such as added complexity of managing dual syringes and purging air from connecting tubing extending between the two syringes.

The art includes various proposals for single syringes including multiple chambers, permitting selective injection of two liquids from the single syringe. However, these various proposals are not readily used in a flexible way as is needed for a typical imaging procedure. For example, many dual-chamber syringes utilize chambers separated along the length of the syringe by a plunger or other divider. Such syringes have the drawback that the two fluids in the syringe may not be selectively injected or mixed in a controllable fashion, since one fluid must flow through the chamber containing the other fluid to reach the patient.

There have been proposals in the art for dual-chamber syringes in which the two chambers are coaxially positioned; that is, the second chamber annularly surrounds the first chamber. This structure has the advantage that the movement and flow of the fluid from the chambers may be independently injected. However, in these known structures, the outer and inner chambers have a connecting point within the body of the syringe, which raises the same difficulties noted in the preceding paragraph, namely, fluids may not be selectively injected or mixed, since there will always be mixing of fluids at the connection point of the chambers. Hence, the present invention allows fluids to be selectively, and independently, injected into a subject or mixed outside of the syringe chambers prior to injection.

### SUMMARY OF THE INVENTION

The present invention is directed to a dual chamber syringe that avoids these difficulties of the prior art, which comprises an inner cylindrical body having a open end, a closed end with an inner discharge outlet formed therein, and an inner plunger sealingly slideable within the inner body, and an outer cylindrical body enclosing the inner cylindrical body and having an open end, a closed end with an outer discharge outlet formed therein, and an outer plunger sealingly slidable between the outer body and the inner body, the inner discharge outlet extending into the outer discharge outlet such that the inner body is in fluid communication through the outer discharge outlet with the exterior of the inner and outer body.

In specific embodiments of the syringe, the syringe includes a backer plate mounted between the open rearward end of the inner and outer bodies to maintain a spacing therebetween. A ram (or pushrod) extends from the inner plunger through the open end of the inner body outside the inner body, and a ram (or pushrod) extends from the outer plunger through an aperture in the backer plate outside the outer body, thus permitting selective movement of the inner and outer plungers for an injection.

An injector for use with the syringe described above includes first and second motorized drives, the first drive engaging to the ram coupled to the inner plunger and the second drive coupled to the ram coupled to the outer plunger, the drives independently driving the inner and outer plungers for injection of fluid.

These and other objectives of the present invention will be more readily apparent from the following detailed description of the drawings.

### BRIEF DESCRIPTION OF THE DRAWINGS

The accompanying drawings, which are incorporated in and constitute a part of this specification, illustrate embodiments of the invention and, together with a general description of the invention given above, and the detailed description of the embodiments given below, serve to explain the principles of the invention.

FIG. 1A is a partial schematic longitudinal section of one embodiment the dual chamber syringe and tip cap.

FIG. 1B is a schematic longitudinal section of the front end of another embodiment of the dual chamber syringe.

FIG. 1C is a schematic longitudinal section of the front end of an additional embodiment of the dual chamber syringe with internal threads in the nozzle.

FIG. 1D is a schematic longitudinal section of the front end of the dual chamber syringe and an adapter.

FIG. 1E is a schematic longitudinal section of the front end of the dual chamber syringe with an adapter secured to the nozzle.

FIG. 1F is a perspective view of a backplate with gripping edges engaged with syringe.

FIG. 1G is a partial schematic longitudinal section of an alternative embodiment of the dual chamber syringe and tip cap.

FIG. 2 is a perspective view of rear section of backplate.

FIG. 3 is a perspective view of dual chamber syringe.

FIG. 3A is a perspective view of the alternative embodiment of the dual chamber syringe of FIG. 1G.

FIG. 4 is a view showing pushrods and drive mechanism.

FIG. 4A is a view showing pushrods and drive mechanism used with the alternative embodiment of the dual chamber syringe of FIG. 1G.

FIG. 5 is a view of a manually operated pushrod device.

FIG. 5A is a view of a manually operated pushrod device used with the alternative embodiment of the dual chamber syringe of FIG. 1G.

FIG. 6 is a view showing an alternative embodiment of pushrods and drive mechanism.

FIG. 7A is a longitudinal section of an outer piston of the alternative embodiment of the dual chamber syringe of FIG. 1G.

FIG. 7B is a perspective view of an outer piston of the alternative embodiment of the dual chamber syringe of FIG. 1G.

FIG. 7C is a perspective view of the engagement of the pistons of the alternative embodiment of the dual chamber syringe of FIG. 1G to the pushrods of FIG. 4A.

FIG. 7D is a perspective view showing the rotation of the alternative embodiment of the dual chamber syringe of FIG. 1G to engage its pistons to the pushrods of FIG. 4A.

FIG. 7E is a perspective view showing the rotation of the pushrods of FIG. 4A to engage the pistons of the alternative embodiment of the dual chamber syringe of FIG. 1G.

### DETAILED DESCRIPTION OF THE INVENTION

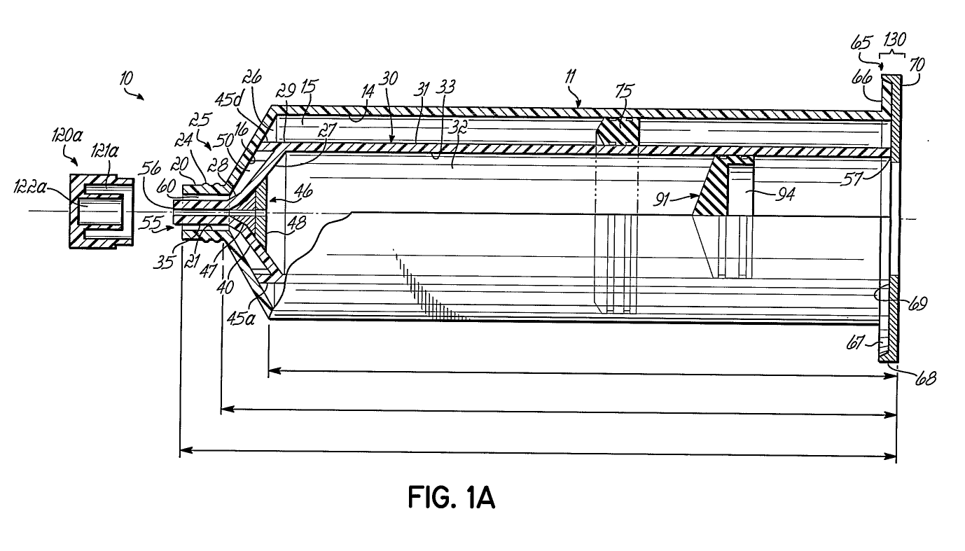

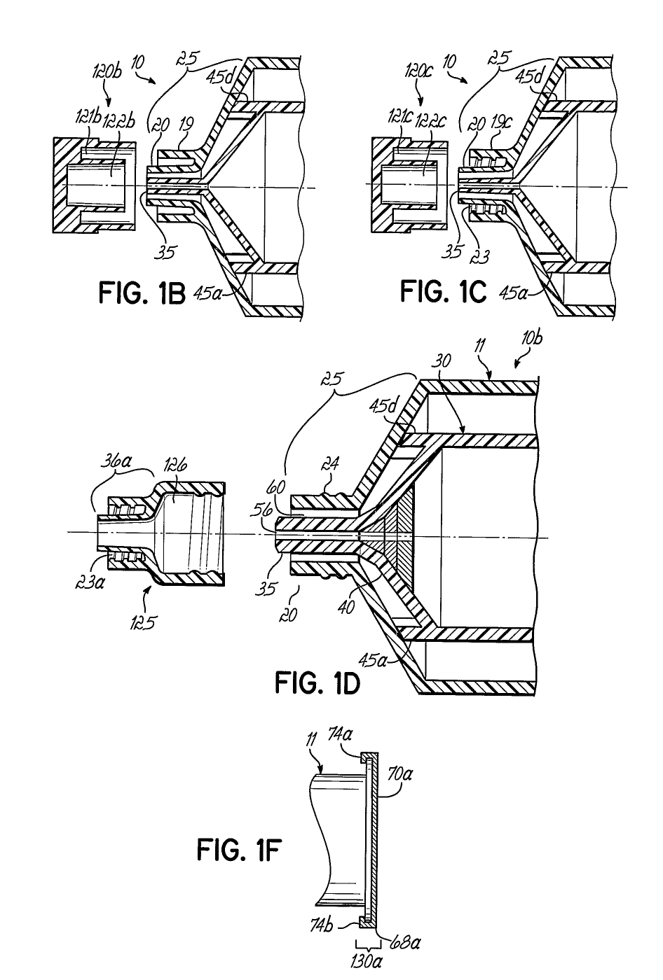

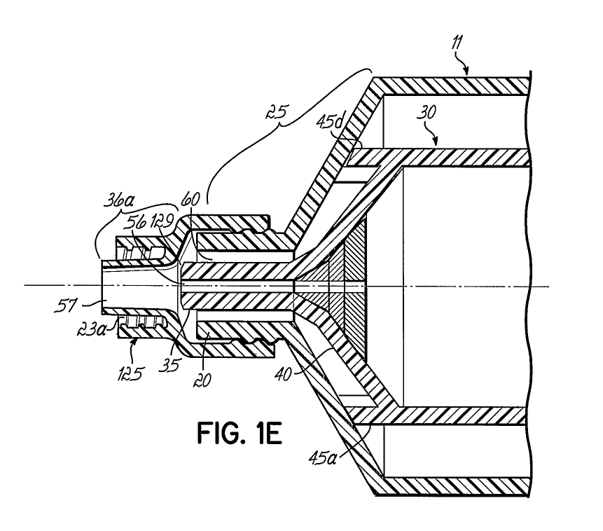

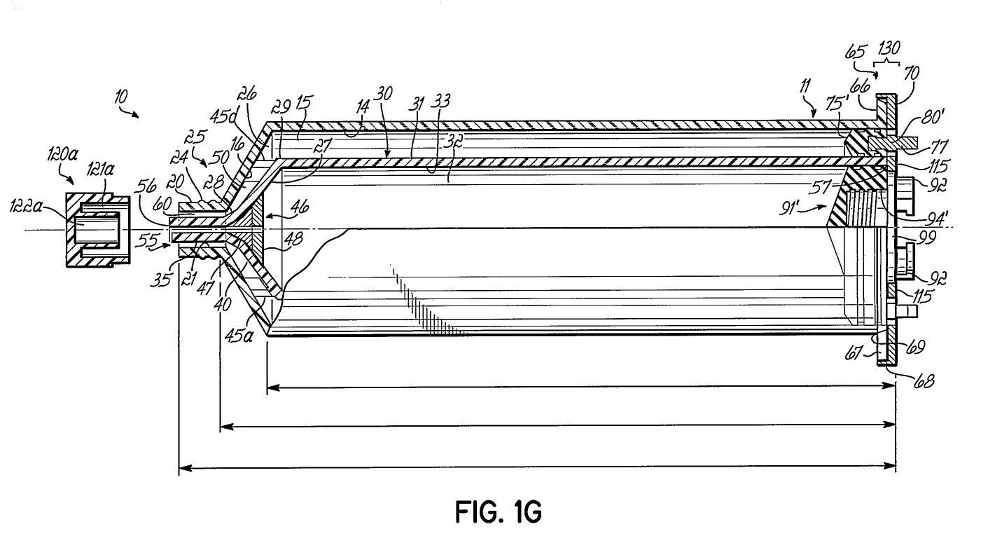

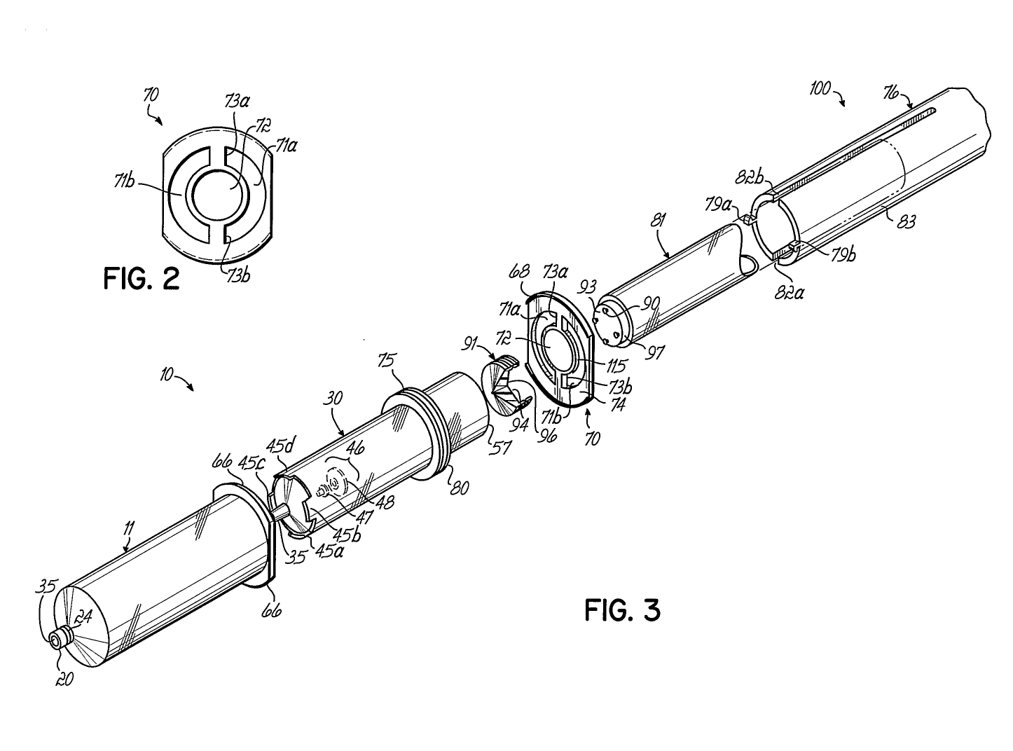

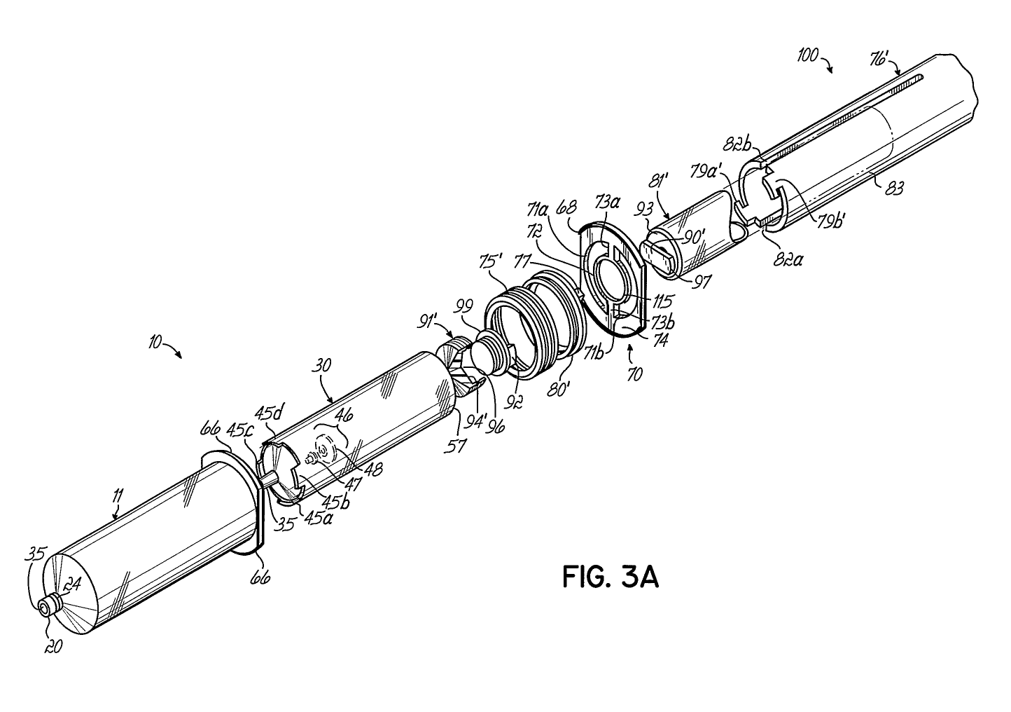

As noted in the Background section, there is a need for a dual chamber syringe, which allows fluids to be selectively, and independently, injected into a patient, or mixed outside of the syringe chambers prior to injection. As shown in FIGS. 1A and 3, the dual chamber syringe 10 of the invention has an outer body 11 whose inner side or wall 14 provides the outer circumferential wall of the outer chamber 15. The syringe 10 has a nozzle 20 extending from the apex of the conical cone shaped forward end or nozzle end 25 of the outer body 11.

Positioned inside the outer body 11 is an inner body 30. The outer wall 31 of the inner body 30 provides the inner circumferential wall of the outer chamber 15 and its inner wall 33 circumferentially encloses the inner chamber 32. The forward end 40 of the inner body 30 has a conical section 27 and a nozzle 35 extending outward from the apex of the conical or cone section 27 of the inner body 30. The inner body 30 has four evenly spaced projection or tabs 45a-d, as shown in FIG. 3, circumferentially extending from the base portion 29 of the conical section 27 that extends from the body 30 and provides a forward chamber 50 that is continuous with the outer chamber 15, FIG. 1A. The projections 45a-d are bonded to the inner surface 16 of the cone shaped forward end 25 of the outer body 11 by methods known to one skilled in the art such as ultra-sound bonding, laser bonding, etc. The nozzle 35 of the inner body 30 is of a size that is insertable into the opening 55 of nozzle 20 of the outer body 11. The diameter of the nozzle 35 is small enough that a first passageway 60 is provided between the outer surface of nozzle 35 of the inner body 30 and inner surface 21 of nozzle 20 that permits the flow of fluid from the outer chamber 15. Nozzle 35 has a second passageway or opening 56 that permits the outflow of fluid from the inner chamber 32.

A tip cap 120a may be inserted over nozzles 20 and 35. As shown in FIG. 1A, the cap 120a may have a circular recess or slot 121a into which nozzle 20 is inserted and cylindrical recessed bore or cavern 122a into which nozzle 35 is inserted.

In another embodiment as shown in FIG. 1B, there is a cylindrical protrusion 19 that extends from the conical forward end 25 that encircles the outer nozzle 20. The tip cap 120b has a circular recess or slot 121b into which protrusion 19 is inserted and a cylindrical barrel shaped recess 122b into which both the inner nozzle 35 and outer nozzle 20 are inserted. In the embodiment shown in FIG. 1C, the inner surface of protrusion 19c may also have threads 23. The top cap 120c has circular recess or slot 121c and cylindrical recessed bore or cavern 122c to engage with the protrusion 19 and nozzles 11 and 35 as described for FIG. 1B. The slot 121c may include threads that mate with threads 23 or may be unthreaded and fit by interference over outer nozzle 20. The syringe tip 20 of all embodiments of the invention meets the leur taper specifications as set forth in the ISO standard.

In other embodiments, the nozzle 20 may optionally have one or more external threads 24 as shown in FIGS. 1A and 1D or internal threads 23 as shown in FIG. 1C. The external threads 24 can engage female Luer locks and the internal threads 23 may engage threads of a male Luer lock (Luer locks not shown). The Luer locks are part of a fluid line connected to a patient for injection of fluids. The syringe 10b shown in FIG. 1D is an enlarged perspective of the syringe 10 in FIG. 1A. Also shown in FIG. 1D is an adapter 125, which can connect to syringe 10b as shown in FIG. 1E. The adapter 125 allows for the connection of a large syringe tip, which does not meet ISO standards, to a small female Luer lock, which complies with ISO standards. The adapter 125 is made of a hard plastic, and has internal threads 126 in a cylindrical extension on one end thereof, which engage the external threads 24 of nozzle 20. The adapter 125 also has internal threads 23a inside a cylindrical extension on its second end, and a male Luer connector tip or nozzle end 36a, which enables the syringe 10b to connect to a female Luer lock. The cylindrical extensions and Luer connectors are coaxial. As shown in FIG. 1E, when the adapter 125 is connected to the nozzle 20 the first passageway 60 opens into third passageway 129, which is formed upon making the connection of adapter 125 and nozzle 20. The second passageway 56 opens into fourth passageway or opening 57 of the adapter 125. These connecting passageways or openings allow fluid to flow from the outer chamber 15 and the inner chamber 32 to the Luer lock connection through the fourth passageway 57 of the adapter 125.

The rearward edge 65 of the outer body 11 has a flange 66 extending radially outward, FIG. 1A. The flange 66 adapts to the circumferential recess 67 formed by a tapered ridge 68, or knurl, on the forward surface 69 of the backer plate 70, 70a. In an alternative embodiment as shown in FIG. 1F, the backer plate 70 assembly has two griping edges 74a, 74b, which secure flange 67 of the outer body 11.

Referring to FIG. 3, the backer plate assembly 70 has two semi-lunate, i.e., crescent-shaped, openings 71a, 71b opposite of each other and positioned a certain distance from a circular opening or aperture 72 as defined by the thickness of the inner girdle or inner circular ring 115. The aperture 72 is formed by the inner surface of inner circular ring 115. The circular ring 115 is integrally connected to the outer disc 74 by bridge members 73a, 73b. Welding, adhesion or other means known to one skilled in the art bonds the inner ring 115 of the backer plate 70, 70a and the end 57 of the inner barrel 30. The outer disc 74 of the backer plate 70, 70a and the flange 66 of the outer barrel 11 are also bonded keeping the inner and outer barrels in a coaxial position. Additionally, the backer plate syringe assembly 130, FIGS. 1A and 1F, prevents the inner barrel from coming off the outer barrel when the inner rubber plunger tip 30 is pulled back.

Inside the outer chamber is a slidable toroidally shaped rubber piston 75 that is in contact with the surface of the inner wall 14 of the outer body 11 and contacts the surface of the outer wall 31 of the inner body 30. When the syringe 10 is mounted on the drive mechanism 105, the pushrod 76 engages the rubber piston 75 by inserting at least two or more hooks or locking projections 79a, b into corresponding slots for engagement on the rear wall 80 of the rubber piston 75 to secure the pushrod 76 to piston 75. Additionally, the cylindrical extension 93 of the inner chamber pushrod 81 has two or more locking type projections 90 on the surface of the cylindrial extension 93 of pushrod 81 that engage corresponding slots in the inner wall 96 of the recess 94 to secure the pushrod 81 with piston 91. In another embodiment the wall 97 of cylindrical extension 93 has threads that engage threads on the wall of recess 94 of piston 91 (threads not shown) to form, a screw lock mechanism may be used to secure the rod 81 to piston 91.

Referring to FIGS. 1G, 3A, 7A and 7B, in an alternative embodiment of the dual chamber syringe shown in the preceding Figs., an alternative outer piston is formed of a rubber cover 75′ over a hard plastic core 80′ forming the rear surface of the outer piston. Integrally formed in the hard plastic backer plate/rear surface 80′ are hooks 77 for engaging hooks or locking projections 79a′ and 79b′ on the front surface of outer pushrod 76′. Further, an alternative inner piston is formed of a rubber cover 91′ over a hard plastic backer plate 99. Backer plate 99 is engaged to threads or other engagement features on the interior of a recess 94′ in the rear side of rubber cover 91′. Backer plate 99 includes hooks 92 for engaging a t-shaped hook 90′ at the outer end of the inner pushrod 81′. Other features of the embodiment of FIG. 1G are similar to those previously described and like reference numerals have been used for like features thereon.

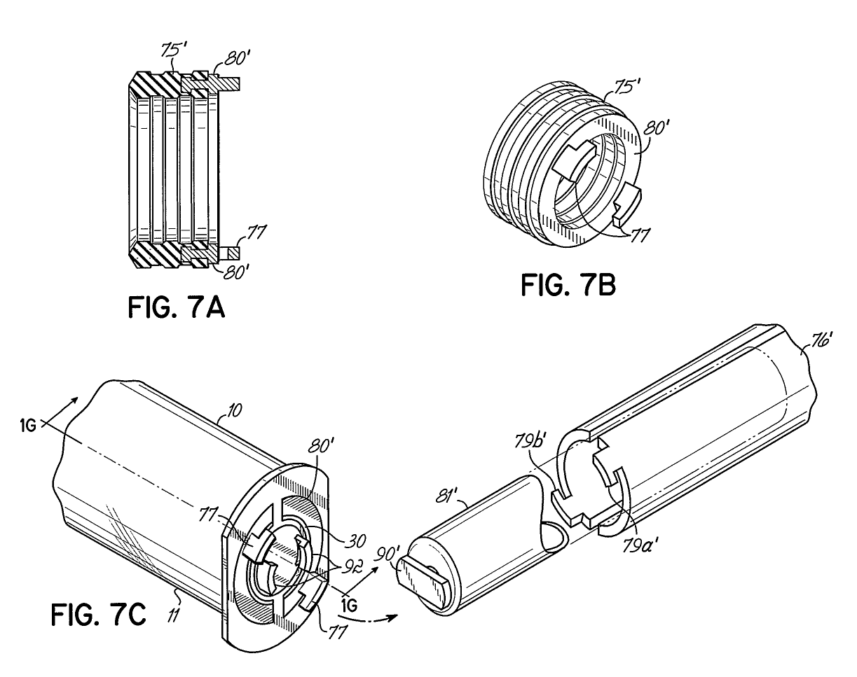

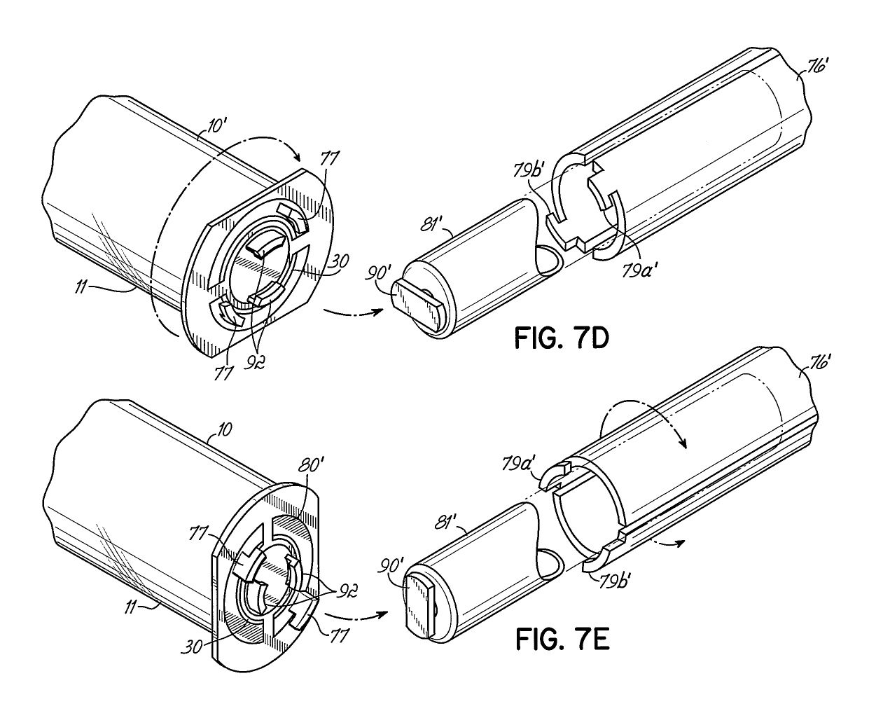

FIG. 7C illustrates the engagement of the hooks 77 on backer plate 80′ and hooks 92 on backer plate 99 to the hooks 79a′ and 79b′ and t-shaped hook on pushrod 81′. Specifically, hooks 77 couple into hooks 79a′ and 79b′ and hooks 92 couple to t-shaped hook 90′ when the syringe 10 and pushrods 76′ and 81′ are relatively oriented as shown in FIG. 7C. In one embodiment, illustrated in FIG. 7D, syringe 10 is installed rotated 90 degrees from the position shown in FIG. 7C and then is rotated as shown by the arrow in FIG. 7D to an engaged position as shown in FIG. 7C. In an alternate embodiment, illustrated in FIG. 7E, pushrods 76′ and 81′ are rotated 90 degrees from the position shown in FIG. 7C and the syringe is installed while the pushrods 76′ and 81′ are in this position, and once the syringe is installed, pushrods 76′ and 81′ are rotated as shown by the arrow in FIG. 7E to an engaged position as shown in FIG. 7C.

Referring to FIGS. 1A and 3, inside the inner chamber is a valve assembly 46 comprised of a one-way valve 47 (not shown in FIG. 1A), commonly known as a duckbill valve and a valve plate 48 to fix valve 47 in the appropriate position. The valve plate 48 is bonded at a position in the conical section 27 as seen in FIG. 1A.

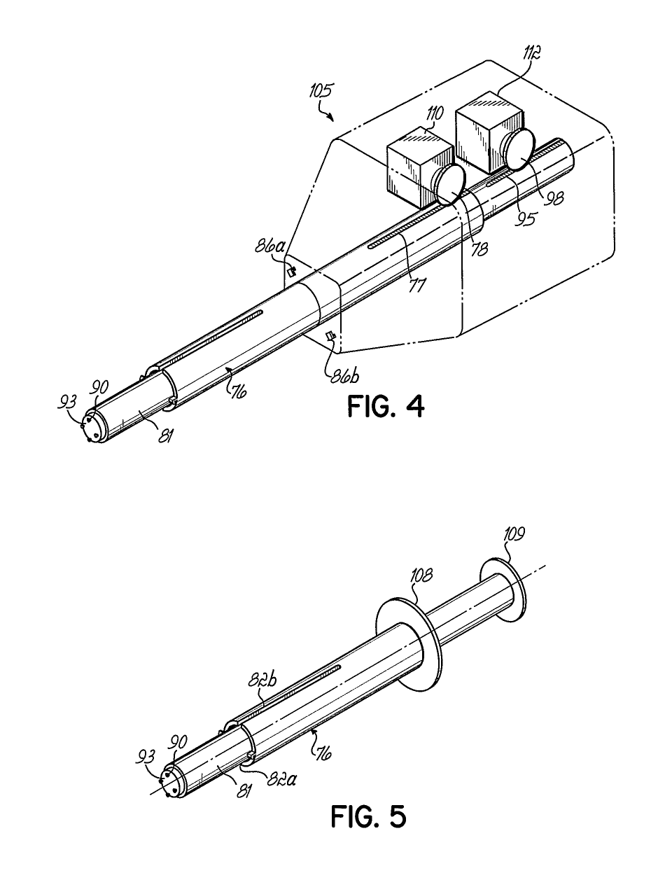

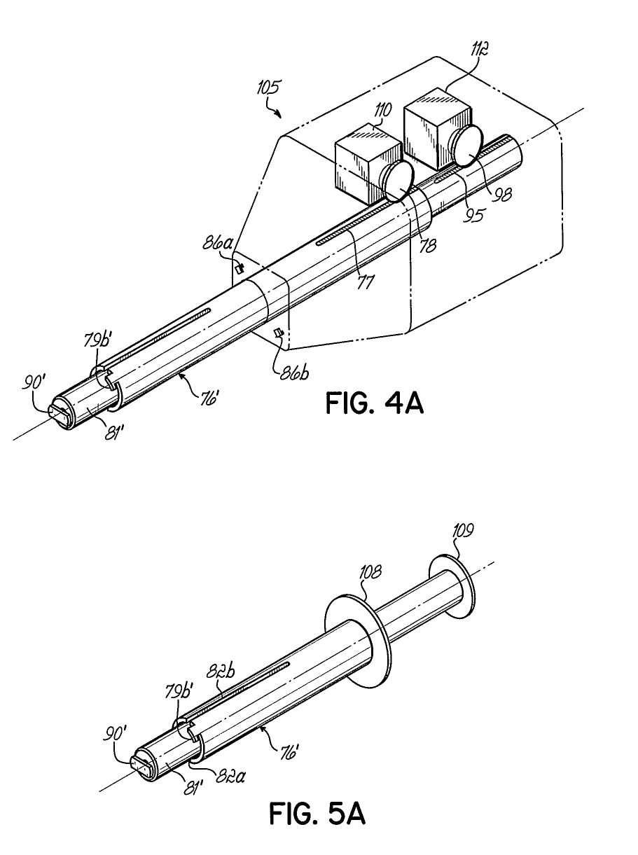

The outer chamber pushrod 76 of the coaxial plunger system 100 is a hollow cylinder having a diameter large enough to allow the insertion, passage, and sliding movement of the inner chamber pushrod 81. Pushrod 76 has two opposed longitudinal slots 82a, 82b along its cylindrical wall 83 that extend along its length from its forward section rearward and terminating a distance from its rear section. The slots 82a, 82b bifurcate the pushrod 76 such that its two halves pass through the apertures 71a, 71b in the backer plate 70. In the drive mechanism 105 of FIG. 4, pushrod 76 has a track of teeth 77 that extend a length along the rear portion of the rod 76. The teeth 77 engage the gear mechanism 78 of the injector drive 110 to move the pushrod 76 to slide the piston 75 forward or rearward. The pushrod 76 and piston 75 are engaged as previously described. The pushrods 76 and 81 may also be operated by other driving means as known to one skilled in the art.

The inner chamber pushrod 81 of the coaxial plunger system 100 is cylindrical with a diameter small enough to be inserted and have slidable movement within the outer chamber pushrod 76. The pushrod 81 has cylindrical extension 93 with a flat surface extending from its forward end. A rubber piston 91 which is slidably moveable within the inner chamber 32 engages the extension 93 of the pushrod 81 by means of a recess 94 in its rearward end as previously described. A track of teeth 95 extends a length along the rearward end of pushrod 81. The teeth 95 engage a gear mechanism 98 of the injector drive 112 to slide the pushrod 81 forward or rearward.

The gears 78 and 98 of the injector drives 110 and 112 are independently controlled. The speed of movement of the pushrods 90 and 76 may be different depending upon the injection conditions that need to be maintained. In one embodiment, the drive mechanism 105 has locking projections 86a, 86b, which engage the backer plate assembly 130, 130a, as the syringe 10 is joined to the drive mechanism 105 in a twisting or rotating motion.

FIG. 4A illustrates the use of an alternative inner pushrod 81′ and outer pushrod 76′ with the embodiment of an injector that is otherwise similar to that shown in FIG. 4.

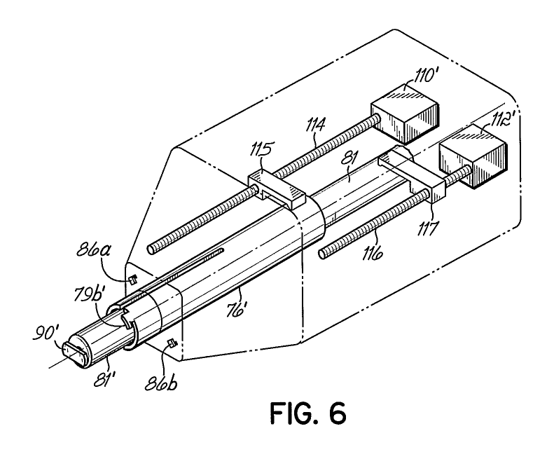

FIG. 6 illustrates an alternative embodiment of driving pushrods which may be used with any embodiment of the pushrods shown herein. In this embodiment the injector drives 110′ and 112′ are coupled to screw shafts 114 and 116 respectively. Followers 115 and 117 are threadedly engaged on screw shafts 114 and 116 and coupled to pushrods 76′ and 81′ respectively. In this embodiment, rotation of drives 110′ and 112′ causes rotation of screw shafts 114 and 116 and translation of followers 115 and 117 along with pushrods 76′ and 81′.

The dual chamber syringe 10 is disposable, and includes walls that will withstand only moderate or low pressure. A pressure jacket is not required in use of this dual chamber syringe assembly. The body of both inner 30 and outer chambers 11 withstand high pressure independently (usually up to about 350 psi pressure). Generally, a contrast media (high viscosity) that requires high pressure is filled in the inner syringe and a saline that requires low pressure is filled in the outer chamber. The pressure resistance of the outer body 11 or barrel is lower than that of the inner body 30 or barrel because the diameter is larger on the same material and thickness. The wall 33 of the inner chamber 32 withstands both expansion and compression pressure. The wall thicknesses of both the inner body 30 and outer body 11 may vary and would be chosen according to the medical solution used and operating pressure, as well as syringe size. As illustrated, the volume of the inner and outer chambers would be approximately 100 ml each, but other volumes are possible; e.g., 50 ml or 200 ml for each chamber.

The syringe 10 may be prefilled with fluids at the factory, or may be filled at a medical services delivery location. Factory pre-filling may fill the syringe inner and outer chambers to various volumes, as desired for a particular medical application. Factory prefilling may be performed with outer and inner bodies 11 and 30 assembled together without pistons 91 and 75, and sealed at their nozzles 20 and 35 by a tip cap. In this procedure, inner body 30 is held in place by a filling tool, and the cavity between inner body 30 and outer body 11 is filled and then plunger 75 is inserted into the cavity. Then inner body 30 is filled and plunger 91 is inserted into inner body 30. Thereafter plungers 75 and 91 are inserted and the backer plate 70 is fixed on the end 57 of inner barrel and flange 66 of outer barrel and the pre-filled syringe is completed. It will be appreciated that the embodiment of the invention shown in FIG. 3A may be assembled empty, and subsequently pre-filled using a filling tool having pushrods similar to those shown in FIG. 3A, to withdraw the plungers 75′ and 91′ to draw fluid through the nozzle 20 into each of the two chambers. Alternately, the embodiment of the invention shown in FIG. 3A may be sold empty and filled at the patient bedside prior to an injection operation.

The dual chamber syringe may be used to administer fluid agents comprising diagnostic agents (e.g., X-ray, magnetic resonance, optical, etc. agents), therapeutic agents, saline and combinations thereof. Both syringes may have the same agent or each chamber may have different agents than the other. Such combinations are, and not limited to, two diagnostic agents, a diagnostic/therapeutic combination, two therapeutic agents, a diagnostic/saline combination and a therapeutic/saline combination. The agents may be injected from each chamber in a sequential manner, that is, first one agent is injected from one chamber then the other agent is injected from the other chamber. The agents may also be injected from the chambers in an alternate or interchangeable manner, that is, a first amount of one agent from one chamber is injected and then a second amount of another agent from the other chamber is injected. This process is repeated as necessary. The agents may also be injected from the chambers simultaneously, that is, the agents are injected at the same time. The rates of injection from both chambers may be independently varied depending upon the diagnostic or therapeutic procedure used in the subjects as known to one skilled in the art. The injector mechanism may be the drive mechanism as previously described and adapted to a power injector, manually operated devices that have inner and outer pushrods to move the pistons of the dual chamber syringe, or any other device that is capable of moving the pistons to inject fluids into a subject or patient.

An example of a manually operated pushrod device is shown in FIG. 5. There is an inner pushrod 81 that is within an outer pushrod 76, both pushrods engage pistons 91 and 75 as previously described for the same described for FIG. 4. At the end of pushrod 76 and pushrod 81, there are extensions 108, 109 that allow the user to manually move the pushrods in the syringe 10. The shape of the extensions 108, 109, may be circular, rectangular, have opposing straight and curved edges or other shapes as known to one skilled in the art to allow the user to move the pushrods 76, 81, within the dual chamber syringe 10.

FIG. 5A illustrates the use of an alternative inner pushrod 81′ and outer pushrod 76′ with the embodiment of a manually operated pushrod device that is otherwise similar to that shown in FIG. 5.

While the present invention has been illustrated by the description of an exemplary embodiment thereof, and while the embodiment has been described in considerable detail, it is not intended to restrict or in any way limit the scope of the appended claims to such detail. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of Applicants' general inventive concept.

1. A medical fluid injector assembly comprising:
a dual chamber syringe comprising:
a cylindrical inner body having an open end, a closed end with an inner discharge outlet formed therein, and an inner plunger sealingly slidable within the inner body, and
a cylindrical outer body enclosing the inner body and having an open end, a closed end with an outer discharge outlet formed therein, and an outer plunger sealingly slidable between the outer body and the inner body, wherein the inner discharge outlet extends into the outer discharge outlet such that the inner body is in fluid communication through the outer discharge outlet with an exterior of the inner and outer bodies; and
a motorized injector on which the syringe is mounted, the injector comprising:
first and second motorized drives designed to independently drive the inner and outer plungers.

2. The assembly of claim 1, further comprising:
a backer plate mounted between the open rearward end of the inner and outer bodies.

3. The assembly of claim 2, further comprising:
a ram extending from the outer plunger through an aperture in the backer plate outside the outer body.

4. The assembly of claim 1, further comprising:
a ram extending from the inner plunger through the open end of the inner body outside the inner body.

5. The assembly of claim 1, wherein the first drive is designed to engage a ram coupled to the inner plunger.

6. The assembly of claim 1, wherein the second drive is designed to engage a ram coupled to the outer plunger.

7. The assembly of claim 1, for use in injecting fluids into a subject, wherein the fluids are contained in an outer chamber and an inner chamber of the syringe, and wherein the assembly is designed such that the fluids in the chambers are injected sequentially, interchangeably, or simultaneously.

8. An injector for a dual chamber syringe comprising:
an inner ram,
a first motorized drive drivingly coupled to the inner ram,
an outer ram comprising a cylindrical section annularly surrounding the inner ram,
a second motorized drive drivingly coupled to the outer ram, and
a housing for mounting of a syringe having an inner housing with an inner plunger therein and an outer housing with an outer plunger therein, such that the inner ram is engageable to drive the inner plunger and the outer ram is engageable to drive the outer plunger.

9. The injector of claim 8, wherein the inner ram comprises a track of teeth, and the first motorized drive comprises a gear meshing with the teeth.

10. The injector of claim 8, wherein the outer ram comprises a track of teeth, and the second motorized drive comprises a gear meshing with the teeth.

11. The injector of claim 8, wherein the inner ram comprises a threaded follower, and the first motorized drive comprises a screw rotatable by the first motorized drive and threadedly engaged to the follower.

12. The injector of claim 8, wherein the outer ram comprises a threaded follower, and the second motorized drive comprises a screw rotatable by the second motorized drive and threadedly engaged to the follower.

13. An adapter for a syringe tip, comprising:
a first end haying a substantially cylindrical extension defining an opening therein, the opening having a threaded interior surface for engagement to a syringe tip that is not compliant with an ISO standard; and
a second end defining an opening therein, the opening comprising a male Luer connector extending outward from the opening, and a substantially cylindrical extension surrounding the male Luer connector and having a threaded interior surface, the male Luer connector and the substantially cylindrical extension of the second end being compliant with an ISO standard, wherein the adapter has a channel defined therein that extends between the first end opening and the male Luer connector to adapt a non-ISO-compliant syringe tip to an ISO standard tubing connector.

14. The adapter of claim 13, wherein the cylindrical extension of the first end is larger in diameter than the cylindrical extension of the second end.

15. The adapter of claim 13, wherein the cylindrical extension of the first, the cylindrical extension of the second end, and the male luer connector are substantially co-axial.

16. The adapter of claim 13, wherein the adapter is manufactured of a hard plastic.
